(19) United States
(12) Patent Application Publication
(10) Pub. No.: US 2014/0255473 A1
(43) Pub. Date: Sep. 11, 2014

(54) STEROID HORMONE DELIVERY SYSTEMS AND METHODS OF PREPARING THE SAME

(71) Applicant: MONOSOL RX, LLC, Warren, NJ (US)

(72) Inventors: Eric Dadey, Furlong, PA (US); Alexander Mark Schobel, Whitehouse Station, NJ (US)

(73) Assignee: MONOSOL RX, LLC, Warren, NJ (US)

(21) Appl. No.: 14/281,890

(22) Filed: May 19, 2014

Related U.S. Application Data
(63) Continuation of application No. 13/588,731, filed on Aug. 17, 2012.
(60) Provisional application No. 61/524,847, filed on Aug. 18, 2011.

(51) Int. Cl. A61K 31/568; A61K 9/70; A61K 9/107; A61K 9/127; A61K 9/14

(52) U.S. Cl. CPC A61K 31/568; A61K 9/127; A61K 9/145; A61K 9/1271; A61K 9/1075; A61K 9/7007
USPC 424/450; 514/181

(57) ABSTRACT

The present invention is directed to steroid hormone delivery systems and methods of preparing the same. In particular, the steroid hormone delivery systems provided include a primary construct having one or more hydrophobic steroid hormone esters in the form of a liposome, a lipid particle, a micelle, an emulsion or a niosome which is then formulated into a secondary construct for administration. Exemplary secondary constructs include a film for sublingual or buccal administration.

### CROSS-REFERENCE TO RELATED APPLICATION

This application is a Continuation of U.S. patent application Ser. No. 13/588,731, filed Aug. 17, 2012 which claims the benefit of U.S. Provisional Application No. 61/524,847, filed Aug. 18, 2011, the contents of which is incorporated by reference herein.

### FIELD OF THE INVENTION

The present invention provides steroid hormone delivery systems and methods of preparing the same. In particular, the steroid hormone delivery systems provided include a primary construct including one or more hydrophobic steroid hormone esters in the form of a liposome, a lipid particle, a micelle, an emulsion or a niosome wherein the primary construct is formulated into a secondary construct for administration. In one embodiment, the secondary construct is in the form of a film suitable for administration to a mucosal surface, e.g., oral, vaginal, rectal, nasal or ocular surfaces. Particularly useful mucosal surfaces are the buccal and sublingual surfaces. In another embodiment, the secondary construct is in the form of a liquid suspension suitable for enteral and/or parenteral administration.

### BACKGROUND OF THE RELATED TECHNOLOGY

There is a need for delivery systems to administer steroid hormones for medicinal indications with favorable pharmacokinetics that foster increased patient compliance and/or provide increased patient comfort during administration thereof. For example, non-injectable formulations for sustained release of steroid hormones are desirable. There is a particular need for delivery systems for steroid hormones which can achieve approximate steady state levels of hormones in the blood relative to prior delivery methods.

### SUMMARY OF THE INVENTION

The present invention provides steroid hormone delivery systems and methods of preparing the same which overcome the problems associated with prior delivery systems. In particular, steroid hormone delivery systems are provided having a primary construct with one or more hydrophobic steroid hormone esters in a liposome, a lipid particle, a micelle, an emulsion or a niosome wherein the primary construct is formulated into a secondary construct having at least one pharmaceutically acceptable excipient. The secondary construct desirably is in the form of a solid dosage or semi-solid form or a liquid dosage form, such as a suspension. Additionally, the present invention provides methods of forming a steroid hormone depot.

Advantageously, such delivery systems exploit the hydrophobic nature of steroid hormones and, in essence, provide a delivery system within a delivery system which has favorable pharmacokinetics upon administration thereof.

### BRIEF DESCRIPTION OF THE FIGURES

FIG. 1 shows a graph of plasma testosterone levels over a 12 hr period in three minipigs following buccal administration of both 8 mg testosterone enanthate (TE) film and 8 mg testosterone undecanoate (TU) film.

FIG. 2 shows a graph of plasma testosterone levels over a period of time in three minipigs following topical administration of 20 mg FORSTESTA® (testosterone gel).

### DETAILED DESCRIPTION OF THE INVENTION

As used herein, the term “construct” means a delivery system for release of active. In the context of the present invention, a “primary” construct refers to a delivery system which is formulated using one or more hydrophobic steroid hormones wherein the primary construct itself is used as a “component” in formulating a “secondary” construct which further includes at least one pharmaceutically acceptable excipient. In other words, a delivery system containing active is itself used as a component to formulate a higher order delivery system.

In one aspect, the present invention provides steroid hormone delivery systems including: a primary construct having one or more hydrophobic steroid hormones and one or more of the following: a lipid, an oil, a polymer, or a surfactant; wherein the primary construct is in the form of a micelle, a liposome, a lipid particle, an emulsion or a niosome; and a secondary construct including the primary construct and at least one pharmaceutically acceptable excipient.

In one embodiment, the primary construct includes a surfactant and the primary construct is in the form of a micelle. In one embodiment, the primary construct includes a polymer and the primary construct is in the form of a micelle. In one embodiment, the primary construct includes a lipid and the primary construct is in the form of a liposome. In one embodiment, the primary construct includes both a lipid and a surfactant, and the primary construct is in the form of a lipid particle. In one embodiment, the primary construct includes both a surfactant and oil, and the primary construct is in the form of an emulsion. In one embodiment, the primary construct includes a surfactant and the primary construct is in the form of a niosome.

In one embodiment, the secondary construct is in the form of a liquid suspension. In one embodiment, the secondary construct is in the form of a film. In one embodiment, the secondary construct is in the form of a liquid dosage form, solid dosage form or semisolid dosage form.

In another aspect, the present invention provides methods of preparing a steroid hormone delivery system including: preparing a primary construct having one or more hydrophobic steroid hormones and one or more of the following: a lipid, an oil, a polymer, or a surfactant; wherein the primary construct is in the form of a micelle, a liposome, a lipid particle, an emulsion or a niosome; and preparing a secondary construct wherein the primary construct and at least one pharmaceutically acceptable excipient is formulated into a dosage form for administration.

In yet another aspect, the present invention provides methods of forming a steroid hormone depot wherein a liquid suspension of the present invention is administered parenterally.

In still yet another aspect, the present invention provides methods of forming a steroid hormone depot wherein a film of the present invention is administered sublingually, buccally, vaginally or rectally.

In some embodiments, the delivery systems of the present invention provide release of steroid hormone for at least 3 hours to about 4 hours, at least 12 hours or at least 24 hours. In one embodiment, the delivery systems provide sustained release of steroid hormone for greater than 7 days, at least 10 days, at least 14 days or at least 21 days.

Suitable hydrophobic steroid hormones include, but are not limited to, testosterone esters for delivery of testosterone. In particular, suitable testosterone esters include, but are not limited to, testosterone enanthate, testosterone cypionate, testosterone undecanoate, testosterone propionate, testosterone formate, testosterone acetate, testosterone butyrate, testosterone valerate, testosterone caproate, testosterone isocaproate, testosterone heptanoate, testosterone octanoate, testosterone nonanoate, testosterone decanoate or a combination of two or more thereof.

Suitable lipids for the preparation of liposomes include, but are not limited to, cholesterol, cholesterol sulfate, phosphatidic acid, phosphatidylcholine, phosphatidylethanolamine, phosphatidylglycerol, phosphatidylserine, lysophosphatidylcholine, phosphatidylinositol, phosphatidylinositol phosphate, phosphatidylinositol bisphosphate, phosphatidylinositol triphosphate, ceramide phosphorylcholine, ceramide phosphorylethanolamine, ceramide phosphorylglycerol or a combination of two or more thereof.

Suitable polymeric compounds for the preparation of polymeric micelles include, but are not limited to, polymeric compounds from the following classes of polymeric compounds: poly(ethylene oxide)-b-poly(propylene oxide)s, poly(ethylene oxide)-b-poly(ester)s, and poly(ethylene oxide)-b-poly(amino acid)s. Also contemplated is the use of a combination of two or more polymeric compounds from either the same polymeric class or a different polymeric class listed above.

Suitable surfactants for the preparation of micelles include, but are not limited to, sodium dodecylsulfate, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monooleate, sorbitan monostearate, sorbitan palmitate, sorbitan monolaurate or a combination of two or more thereof.

Suitable oils for the preparation of emulsions include, but are not limited to, liquid paraffin, vegetable oil, olive oil, avocado oil, almond oil, castor oil, sesame oil, jojoba oil, wheatgerm oil, sunflower oil, mineral oil, isopropyl myristate, or a combination of two or more thereof.

Emulsions (e.g., microemulsions and nanoemulsions) include one or more surfactants and one or more oils. Additionally, one or more co-surfactants may optionally be employed. Suitable components for preparing emulsions include, but art not limited to, one or more surfactants, one or more oils and one or more optional co-surfactants listed below in Table 1.

TABLE 1

| SURFACTANT | OIL | COSURFACTANT |
|---|---|---|
| Sodium dodecylsulfate | Liquid Paraffin, | 1-Butanol |
| Polyoxyethylene (20) | Vegetable Oil | 1-Pentanol |
| sorbitan monolaurate | | |
| Polyoxyethylene (20) | Olive Oil, | Diethyleneglycol |
| sorbitan monopalmitate | | monoethyl ether |
| Polyoxyethylene (20) | Almond Oil, | |
| sorbitan monostearate | | |
| Polyoxyethylene (20) | Avocado Oil, | |
| sorbitan monooleate | | |
| Sorbitan monooleate | Jojoba Oil, | |
| Sorbitan monostearate | Wheatgerm Oil, c | |
| Sorbitan palmitate | Castor Oil, | |
| Sorbitan monolaurate | Sunflower Oil | |
| | Sesame Oil | |
| | Mineral Oil | |
| | Isopropyl Myristate | |

Lipid particles include one or more lipids and one or more surfactants. Suitable components for the preparation of lipid particles include, but are not limited to, one or more lipids and one or more surfactants listed below in Table 2.

TABLE 2

| LIPID | SURFACTANT |
|---|---|
| Glyceryl Monosterate | Poloxamer 188 |
| Glyceryl Gistearate | Poloxamer 407 |
| Stearic Acid | |
| Glyceryl Behanate | |

Suitable routes of administration of the drug delivery system include, but are not limited to, oral, buccal, sublingual, parenteral, intravenous, intramuscular, subcutaneous, transdermal, intraperitoneal, intraocular, nasal, inhalational, topical, vaginal or rectal.

Suitable dosage forms include, but are not limited to, liquid dosage forms, solid dosage forms and semisolid dosage forms. In one embodiment, the secondary construct is in the form of a liquid dosage form, solid dosage form or semisolid dosage form.

Exemplary suitable dosage forms include films, pills, tablets, capsules, liquid suspensions (e.g., for oral, ocular, nasal or inhalatory administration or for parenteral injection), flakes, powders, creams, suppositories, and transdermal patches.

Exemplary methods of preparing film delivery systems are described in U.S. Pat. Nos. 7,357,891, 7,897,080, 7,666,337, 7,824,588 and 7,910,031 and Published U.S. Patent Application Nos. US 2011/00033542 and US 2011/00033541, the contents of each of which are incorporated herein by reference in their entirety. Additionally, exemplary methods of preparing pharmaceutical dosage forms are described in Remington: The Science and Practice of Pharmacy. 21st Edition. Philadelphia, Pa. Lippincott Williams & Wilkins (2005), the contents of which are incorporated herein by reference in its entirety. Notably, suitable pharmaceutically acceptable excipients depend on the dosage form being prepared and are also described in Remington: The Science and Practice of Pharmacy (supra).

Though not meant to be limited by any theory with the subject invention, it is anticipated that, when the drug delivery system of the present invention is applied to the sublingual or buccal mucosal surface, the primary construct will absorb into the mucosal tissue and will release the steroid hormone in the aqueous environment of the mucosal tissue. The first construct provides a stabilized form of the hormone and permits incorporation of the hormone into an appropriate second construct, e.g., a delivery system or dosage form, such as a film, which further permits travel across the mucosal membrane. In the case of buccal administration for example, a film may carry the first construct and preferentially release the first construct into and through the mucosa of the buccal tissue. The deposition of the first construct into the tissue may form a type of reservoir or depot in the tissue. Desirably, the first construct preferentially falls apart, or dissociates the hormone, e.g., precipitates out of the first construct into the surrounding tissue, allowing for the slow and continued release into the bloodstream. This release coupled with the hydrophobic nature of the steroid hormone is believed to result in the deposition of the steroid hormone as a “solid” substance in the mucosal tissue. This depot of steroid hormone is then slowly dissolved and absorbed into the systemic circulation. It is believed that steady-state plasma levels of steroid hormone are achievable for at least several hours including at least 24 hours following administration of a single dose. Likewise, it is believed that parenteral administration of the drug delivery system of the present invention is similarly conducive for depot formation.

The features and advantages of the present invention are more fully shown by the following examples which are provided for purposes of illustration, and are not to be construed as limiting the invention in any way.

### EXAMPLES

### Primary Construct

The primary construct includes one or more hydrophobic steroid hormone esters in a liposome, a lipid particle, a micelle, an emulsion or a niosome. Although testosterone ester is exemplified in the following primary constructs, other hydrophobic steroid hormone esters may supplement or be substituted for testosterone ester.

### Micelles

One procedure for preparing a micellar formulation of one or more testosterone esters is to first suspend a known amount of one or more testosterone esters in a volume of water with constant stifling. A solution of surfactant and/or polymeric material is prepared in water. Small aliquots of the surfactant and/or polymeric solution are added periodically to the suspension of testosterone ester(s) with constant stifling. The resulting solution is inspected after each addition and subsequent aliquots are added just until all of the testosterone ester(s) dissolve as indicated by the solution being visually clear.

Alternatively, the components are reversed but result in the formation of a similar micellar formulation. Specifically, a solution is prepared by dissolving a known amount of surfactant and/or polymer in water. Small aliquots of powder testosterone ester(s) are added to the solution with constant stirring to produce a visually clear solution. Additional aliquots of powder testosterone ester are added periodically until the solution exhibits a permanent cloudy, opalescent, or turbid appearance.

Using either of the aforementioned procedures, the optimum ratio of surfactant and/or polymer to testosterone ester(s) is that where the maximum amount of testosterone ester(s) is solubilized by the minimum amount of surfactant and/or polymer.

### Liposomes

In general, liposomal formulations of therapeutics agents are prepared in a multi-step procedure. In the first step, known amounts of lipids (e.g., cholesterol), phospholipids (e.g., phosphatidylcholine, phosphatidylethanolamine) and testosterone ester(s) are dissolved in ethanol. The resulting solution is added to a round-bottom flask and ethanol removed by rotary evaporation. This process results in deposition of the lipids, phospholipids and testosterone ester(s) as a thin layer coating the inside surface of the round bottom flask.

The lipid/testosterone layer is then hydrated with water alone or an aqueous solution containing any combination of salts, pH modifiers, preservatives (e.g., antimicrobial agents), or other stabilizing additives. This hydration process produces liposomal vesicles of various sizes (e.g., diameters approaching 10 to 20 μm) each containing an aqueous core with testosterone ester intercalated into the lipid bilayer.

Mechanical stress (e.g., sonication, extrusion, microfluidization) is then applied to the liposome-containing testosterone suspension in order to reduce and generate liposomes of uniform size.

### Lipid Particles

Lipid particles containing testosterone ester(s) can be prepared by using an emulsification technique. According to this method, a mixture of lipid (e.g., glycerol behenate), surfactant (e.g., poloxamer 407) and testosterone ester(s) is heated to an elevated temperature sufficient to melt the oil and dissolve the testosterone ester(s). Water heated to the same temperature as the oil/surfactant/testosterone ester(s) mixture is added slowly and the resulting dispersion emulsified using a mixer operating at several thousand rpms. The emulsion produced is cooled to room temperature with constant stifling until solidification resulting in the production of lipid microparticles.

### Niosomes

The preparation of niosomes-containing testosterone esters is comparable to the preparation of liposomes. The primary difference is that niosomes use synthetic, non-ionic surfactants (e.g., dialkyl polyglycerol ethers) rather than naturally derived phospholipids to form the lipid bilayer of the vesicles. Otherwise, similar methods are employed. In particular, components are dissolved in a solvent, the solvent is evaporated and the dried components are hydrated followed by exposure to mechanical stress can be used to form niosome-containing testosterone esters of uniform size.

### Emulsions

Emulsions containing testosterone ester(s) can be prepared by dispersing one liquid into another. For example, emulsions can be prepared by mixing, at several thousand rpms, testosterone ester(s), one or more surfactants, one or more oils, water and optionally one or more co-surfactants. Microemulsions and nanoemulsions refer to the size of the particles dispersed therein.

Tables 3 and 4 provide specific examples (formulation compositions) of films that contain a testosterone ester formulated as a microemulsion within the film. The example in Table 3 uses testosterone enanthate as the testosterone ester, whereas the three examples detailed in Tables 4A, 4B and 4C, respectively, use testosterone undecanoate as the testosterone ester. These types of microemulsions are often referred to in the scientific literature as self-emulsfying drug delivery systems (SEDDS).

TABLE 3
11.11 mg Testosterone Enanthate (C111) Formulation*

| | 11.11 mg Testosterone Enanthate (C111) | |
|---|---|---|
| | Formulation with Etocas 35/Transcutol | |
| Component | HP/Capryol 90 | |
| HPMC E15 Dow Lot | 25.3110% (11.390 mg) | |
| U129012N22 and | | |
| YB12012N21 | | |
| PEO WSR N80 LEO | 12.6555% (5.695 mg) | |
| Colorcon | | |
| VBN: ZA 1455S5I1 | | |
| Maltitol added as Lycasin | 12.6555% (5.695 mg) | |
| 80/55 Roquette Batch ULLNB | | |
| and KEGXW | | |
| Testosterone Enanthate | 24.6890% (11.110 mg) | |
| (C111) Lot 035F06631V | | |
| Etocas 35 NF (Cremophor | 10.6160% (4.777 mg) | |
| EL) Croda Lot P-2555 | | |
| Transcutol HP Gattefosse | 1.7290% (0.778 mg) | |
| Lot 450725015 | | |
| Caprylol 90 Gattefosse | 12.3440% (5.555 mg) | |
| Lot 118148 | | |
| % Solids | 30 | |
| % Moisture | 1.38 | |
| Dry Target Strip Weight | 45 | mg |
| Target Strip Weight to | 45.63 | mg |
| Account for % Moisture | 13 × 22 | mm |
| Strip Size | | |

*11.11 mg Testosterone enanthate (C111) is equivalent to 8 mg testosterone base

TABLE 4A
12.67 mg Testosterone Undecanoate (C111) Film Formulations*

| | 12.67 mg Testosterone Undecanoate | |
|---|---|---|
| | Formulation Using Capryol 90/Gelucire | |
| Component | 50/13 System | |
| HPMC E15 | 31.214% (23.410 mg) | |
| Dow Lot YB12012N21 | | |
| PEO WSR N80 LEO | 15.607% (11.705 mg) | |
| Colorcon | | |
| VBN: ZA1455S5I1 | | |
| Sucralose | 2.000% (1.500 mg) | |
| EMD Lot K40112794 919 | | |
| Peceol | 0.500% (0.375 mg) | |
| Gattefosse Batch 127062 | | |
| Testosterone Undecanoate | 16.893% (12.670 mg) | |
| Lot TTUM11001K | | |
| Gelucire 50/13 | 16.893% (12.670 mg) | |
| Gattefosse Lot 106058 | | |
| Capryol 90 | 16.893% (12.670 mg) | |
| Gattefosse Lot 118148 | | |
| % Solids | 30 | |
| % Moisture | 1.57 | |
| Dry Target Strip Weight | 75 | mg |
| Target Strip Weight to Account | 76.196 | mg |
| for Moisture Content | | |
| Strip Weight Range | 74 to 83 | mg |
| Strip Size | 22 × 20 | mm |

*12.67 mg Testosterone Undecanoate (C111) is equivalent to 8 mg Testosterone base

TABLE 4B
12.67 mg Testosterone Undecanoate (C111) Film Formulations*

| | |
|---|---|
| | 12.67 mg Testosterone Undecanoate |
| | Formulation Using Lauroglycol 90/ |
| Component | Gelucire 50/13 System |

| | |
|---|---|
| HPMC E15 | 31.214% (23.410 mg) |
| Dow Lot YB12012N21 | |
| PEO WSR N80 LEO | 15.607% (11.705 mg) |
| Colorcon | |
| VBN: ZA1455S5I1 | |
| Sucralose | 2.000% (1.500 mg) |
| EMD Lot K40112794 919 | |
| Peceol | 0.500% (0.375 mg) |
| Gattefosse Batch 127062 | |
| Testosterone Undecanoate | 16.893% (12.670 mg) |
| Lot TTUM11001K | |
| Gelucire 50/13 | 16.893% (12.670 mg) |
| Gattefosse Lot 106058 | |
| Lauroglycol 90 | 16.893% (12.670 mg) |
| Gattefosse Batch 129653 | |
| % Solids | 30 |
| % Moisture | 0.81 |

| | | |
|---|---|---|
| Dry Target Strip Weight | 75 | mg |
| Target Strip Weight to Account | 75.612 | mg |
| for Moisture Content | | |
| Strip Weight Range | 72 to 80 | mg |
| Strip Size | 22 × 20 | mm |

*12.67 mg Testosterone Undecanoate (C111) is equivalent to 8 mg Testosterone base

TABLE 4C
12.67 mg Testosterone Undecanoate (C111) Film Formulations*

| | 12.67 mg Testosterone Undecanoate | |
|---|---|---|
| Component | Formulation Using Gelucire 50/13 System | |
| HPMC E15 | 31.214% (23.410 mg) | |
| Dow Lot YB12012N21 | | |
| PEO WSR N80 LEO | 15.607% (11.705 mg) | |
| Colorcon | | |
| VBN: ZA1455S5I1 | | |
| Sucralose | 2.000% (1.500 mg) | |
| EMD Lot K40112794 919 | | |
| Peceol | 0.500% (0.375 mg) | |
| Gattefosse Batch 127062 | | |
| Testosterone Undecanoate | 16.893% (12.670 mg) | |
| Lot TTUM11001K | | |
| Gelucire 50/13 | 33.786% (25.340 mg) | |
| Gattefosse Lot 106058 | | |
| % Solids | 25 | |
| % Moisture | 0.78 | |
| Dry Target Strip Weight | 75 | mg |
| Target Strip Weight to | 75.590 | mg |
| Account for Moisture Content | | |
| Strip Weight Range | 72 to 81 | mg |
| Strip Size | 22 × 20 | mm |

*12.67 mg Testosterone Undecanoate (C111) is equivalent to 8 mg Testosterone base

As part of the preclinical evaluation of these testosterone ester formulations, the pharmacokinetic profile of the testosterone enanthate prototype identified in Table 3 and the testosterone undecanoate identified in Table 4C were compared to the pharmacokinetic profile of FORTESTA® (testosterone gel) in minipigs.

Briefly, on Day 1, three (3) castrated Gottingen minipigs were anesthetized, the oral cavity was exposed and the enanthate film was placed on the buccal mucosa and the undecanoate film was placed on the opposite buccal surface of each pig. That is, each pig had two films applied to the oral mucosa. Each film was formulated with a nominal testosterone dose of 8 mg; therefore, the total dose that each pig received was 16 mg testosterone.

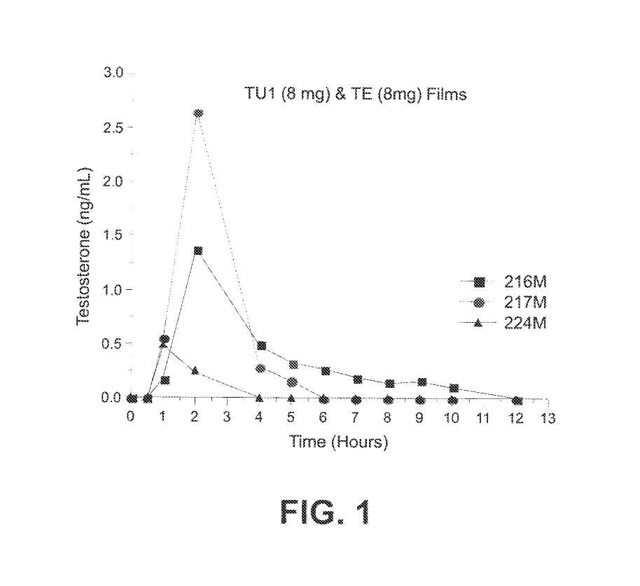

Blood samples were collected periodically over 12 hours and the plasma analyzed for testosterone using an HPLC-MS/MS analytical method. The pharmacokinetic profile of each pig is shown in FIG. 1.

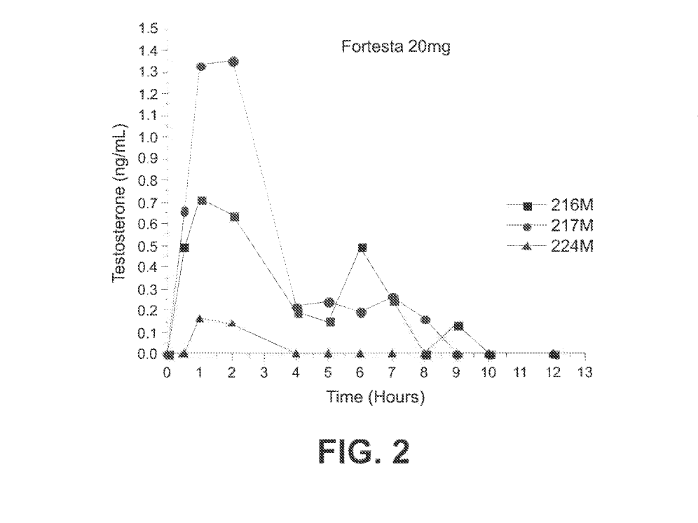

In a control study, the same three minipigs were dosed with 20 mg FORTESTA® (testosterone) topical gel. Blood samples were also collected periodically over 12 hours from each animal and the plasma analyzed for testosterone using an HPLC-MS/MS analytical method. The pharmacokinetic profile of each pig is shown in FIG. 2.

A comparison of the pharmacokinetic profiles of the buccal Testosterone ester films (FIG. 1) to the pharmacokinetic profiles of topical FORTESTA® (testosterone) gel (FIG. 2) shows that both dosage forms provide sustained delivery of testosterone for a minimum of 8 hours. In fact, in Animal Number 216M, the buccal films provide detectable levels of testosterone for at least 10 hours post dosing, whereas no animal in the FORTESTA® (testosterone gel) group showed detectable testosterone levels beyond 10 hours.

Of equal importance is the fact that the total exposure to the drug substance is lower for the buccal films (16 mg) as compared to the topical gel (20 mg). Taken together, the results suggest that the buccal films may provide a therapeutic effect similar to FORTESTA® (testosterone gel) using a lower overall dose.

### Secondary Construct

The secondary construct is prepared using the primary construct as an “ingredient” in formulating a dosage form suitable for administration. A skilled artisan of pharmaceutical formulations can readily adapt conventional techniques for formulating pharmaceutical dosage forms to employ a primary construct of the present invention as an ingredient therein. Importantly, the primary construct may be used in conjunction with the same active found in the primary construct or a different active. Alternatively, the primary construct may be the sole source of active in the dosage form. Also, additional additives may be employed to increase the stability of the steroid hormone delivery system.

Notably, a secondary construct in the form of a film can be prepared using the primary construct as an active ingredient during the preparation of the film. For example, a secondary construct in the form of a film can be prepared using the primary construct as one “ingredient” in the mixture that is used to cast PharmFilm® (MonoSol Rx, Warren, N.J.).

What is claimed is:

1. A steroid hormone delivery system comprising:
a primary construct comprising one or more hydrophobic steroid hormones and one or more of the following: a lipid, an oil, a polymer, or a surfactant; wherein the primary construct is in the form of a micelle, a liposome, a lipid particle, an emulsion or a niosome; and
a secondary construct comprising the primary construct and at least one pharmaceutically acceptable excipient.

2. The steroid hormone delivery system of claim 1, wherein one or more hydrophobic steroid hormone is a testosterone ester.

3. The steroid hormone delivery system of claim 1, wherein the testosterone ester is selected from testosterone enanthate, testosterone cypionate, testosterone undecanoate, testosterone propionate, testosterone formate, testosterone acetate, testosterone butyrate, testosterone valerate, testosterone caproate, testosterone isocaproate, testosterone heptanoate, testosterone octanoate, testosterone nonanoate, testosterone decanoate or a combination of two or more thereof.

3. The steroid hormone delivery system of claim 1, wherein the polymer is selected from poly(ethylene oxide)-b-poly(propylene oxide)s, poly(ethylene oxide)-b-poly(ester)s, and poly(ethylene oxide)-b-poly(amino acid)s.

4. The steroid hormone delivery system of claim 1, wherein the surfactant is selected from sodium dodecylsulfate, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monooleate, sorbitan monostearate, sorbitan palmitate, sorbitan monolaurate or a combination of two or more thereof.

5. The steroid hormone delivery system of claim 1, wherein the lipid is selected from cholesterol, cholesterol sulfate, phosphatidic acid, phosphatidylcholine, phosphatidylethanolamine, phosphatidylglycerol, phosphatidylserine, lysophosphatidylcholine, phosphatidylinositol, phosphatidylinositol phosphate, phosphatidylinositol bisphosphate, phosphatidylinositol triphosphate, ceramide phosphorylcholine, ceramide phosphorylethanolamine, ceramide phosphorylglycerol or a combination of two or more thereof.

6. The steroid hormone delivery system of claim 1, wherein the primary construct comprises a surfactant and wherein the primary construct is in the form of a micelle.

7. The steroid hormone delivery system of claim 1, wherein the primary construct comprises a polymer and wherein the primary construct is in the form of a micelle.

8. The steroid hormone delivery system of claim 1, wherein the primary construct comprises a lipid and wherein the primary construct is in the form of a liposome.

9. The steroid hormone delivery system of claim 1, wherein the primary construct comprises both a lipid and a surfactant, and wherein the primary construct is in the form of a lipid particle.

10. The steroid hormone delivery system of claim 1, wherein the primary construct comprises both a surfactant and oil, and wherein the primary construct is in the form of an emulsion.

11. The steroid hormone delivery system of claim 1, wherein the primary construct comprises a surfactant and wherein the primary construct is in the form of a niosome.

12. The steroid hormone delivery system of claim 1, wherein the secondary construct is in the form of a liquid suspension.

13. The steroid hormone delivery system of claim 1, wherein the secondary construct is in the form of a film.

14. The steroid hormone delivery system of claim 1, wherein the secondary construct is in the form of a liquid dosage form, solid dosage form or semisolid dosage form.

15. A method of preparing a steroid hormone delivery system comprising:
preparing a primary construct comprising one or more hydrophobic steroid hormones and one or more of the following: a lipid, an oil, a polymer, or a surfactant; wherein the primary construct is in the form of a micelle, a liposome, a lipid particle, an emulsion or a niosome; and
preparing a secondary construct wherein the primary construct and at least one pharmaceutically acceptable excipient is formulated into a dosage form for administration.

16. A method of forming a steroid hormone depot comprising parenteral administration of the liquid suspension of claim 12.

17. A method of forming a steroid hormone depot comprising sublingual administration of the film of claim 13.

18. A method of forming a steroid hormone depot comprising buccal administration of the film of claim 13.

19. A method of forming a steroid hormone depot comprising vaginal administration of the film of claim 13.

20. A method of forming a steroid hormone depot comprising rectal administration of the film of claim 13.
